(19) United States
(12) Patent Application Publication
(10) Pub. No.: US 2017/0290763 A1
(43) Pub. Date: Oct. 12, 2017

(54) COMPOSITION WITH EXOGENOUS MITOCHONDRIA AS ACTIVE INGREDIENTS AND USE THEREOF AND CELL REPAIRING METHOD THEREFOR

(71) Applicant: TAIWAN MITOCHONDRION APPLIED TECHNOLOGY CO., LTD., Zhubei City, Hsinchu County (TW)

(72) Inventors: Hong-Lin SU, Taichung City (TW); Hsueh-Min TSENG, Taoyuan City (TW); Shih-Fang WU, Taichung City (TW)

(73) Assignee: TAIWAN MITOCHONDRION APPLIED TECHNOLOGY CO., LTD., Zhubei City, Hsinchu County (TW)

(21) Appl. No.: 15/516,010

(22) PCT Filed: Sep. 30, 2014
(86) PCT No.: PCT/CN2014/087975

§ 371 Date: Mar. 30, 2017

(51) Int. Cl. A61K 8/98; A61Q 19/08; A61K 35/407

(52) U.S. Cl. CPC A61K 8/981; A61K 35/407; A61Q 19/08; A61K 2800/74

(57) ABSTRACT

A composition with exogenous mitochondria as active ingredients, and a use thereof and a cell repairing method therefor. The composition includes exogenous mitochondria and at least one pharmaceutically or cosmetically acceptable carrier. The composition may further include an adjuvant, and the adjuvant is selected from a group consisting of serum, plasma, complement and at least the above two ingredients. The exogenous mitochondria are obtained from cells by a centrifugal purification method.

### CROSS-REFERENCE TO RELATED APPLICATIONS

This non-provisional application is the U.S. National Stage of International

Application No. PCT/CN2014/087975, filed on Sep. 30, 2014, published in Chinese, the entire contents of which are hereby incorporated by reference.

### TECHNICAL FIELD

The disclosure relates to a method for anti-aging and repairing cells with damaged mitochondria, more particular to a composition with exogenous mitochondria as active ingredients, and use thereof and cell repairing method therefor.

### BACKGROUND

Mitochondria in cells are responsible for providing energy to the cells and generating adenosine triphosphate (ATP). The mitochondria perform dynamic deformation according to energy requirement difference or cell stress difference within the cells so that the mitochondria are not always in a single mitochondrion state. Specifically, when the energy requirement of the cell is increased, the mitochondria perform fission continuously so as to generate ATP rapidly. On the other hand, when the cell is in starvation, the mitochondria perform fusion for decreasing energy generation and consumption so as to maintain normal Physiological function. In addition, when the mitochondria are suffer from damages such as the membrane potential drop and the mitochondrial DNA (mtDNA) mutation, etc., the mitochondria also perform fusion for replacing the damaged mtDNA by homologous recombination; when there are to many mutated mtDNA accumulated in the mitochondria, and the mitochondria cannot be repaired, the mitochondria with mutated mtDNA are removed by autophagosome, and only the normal mitochondria are kept (Lamb C A et al., 2013). As a result, when there are to many damaged mitochondria in the cell at the same time and cannot be removed, the cell is drove to cell apoptosis (Mukhopadhyay S et al., 2014).

The mitochondria defect and insufficiency are related to many diseases, such as Leber's hereditary optic neuropathy, mitochondrial enephalomyopathy, lactic acidosis, and strokelike episodes (MELAS), myoclonic epilepsy associated with ragged-red fibers (MERRF), etc. In addition, the neurodegenerative diseases, such as Huntington's disease, Alzheimer's disease, Parkinson's disease, etc., are related to the disorder of the mitochondria fission and fusion (Ghavami S et al., 2014). In addition, the aging phenomena or the age also increases and accumulates the amount of the mutated mtDNA in the mitochondria, which cause the generation of the related disease, such as age-related macular degeneration (AMD) (Brennan L A et al., 2014; Jarrett S G et al., 2010), the skin aging, etc. (Blatt T et al., 2005; Makrantonaki E et al., 2007).

In order to retarding the skin aging, people use many cosmetic products, such as hyaluronic acid, vitamin A and vitamin C, antioxidants, sunscreens, etc., or seek medical beauty clinics for reducing the aging phenomena, such as laser, botulinum, radio frequency skin tightening, etc. However, the conventional cosmetic method cannot reduce or retard the occurrence of skin aging. Another research confirms that injecting mesenchymal stem cells is able to effectively reduce the aging phenomena such as skin wrinkle, and therefore, the stem cell therapy is considered as an opportunity to retard skin aging. However, actually, stem cells are difficult to be obtained, and the massive cultivation of stem cells takes too much time and money. Moreover, stem cell transplantation has the risk such as mutations which produces tumors or rejection. As a result, there is no method with high safety today for effectively reducing the skin aging.

The research in 1989 shows when providing exogenous mitochondria to cells for co-culturing, the exogenous mitochondria are able to enter the cells after direct injection or membrane fusion, and therefore, the cells with the mitochondria having gene defect restore normal function (King M P et al., 1988; King M P et al., 1989). Many researches also confirm that simply co-culturing the cells and the mitochondria is not able to make the mitochondria enter the cells (Chang J C et al., 2013; Spees J L et al., 1989), and therefore, whether the exogenous mitochondria, which are directly provided to the cells, enter the cells is not able to be confirmed. According to the prior art, the abilities of different cells for exogenous mitochondria intake may be different. Therefore, as the route for the mitochondria to enter the cells has not been figure out, the researcher cannot repeat the experiment regarding to the mitochondria by adjusting the experiment condition.

Moreover, although the cells are able to engulf exogenous substances, such as bacteria, etc., by phagocytosis, but the exogenous substances engulfed by phagocytosis form the phagolysosome with the lysosome so that the exogenous substances are degraded. As a result, it is generally believed that the exogenous mitochondria are not able to be kept in the cell by phagocytosis and repair the endogenous mitochondria. Recent researches use the cell penetrating peptide disclosed in U.S. Pat. No. 8,648,034 or the liposomal coating mitochondria disclosed in U.S. Pub. No. 2013/0022666 to help the mitochondria to fuse with the cell membrane, and the mitochondria get into the cell easily for improving the oxidative respiration of the cell. However, although the above methods are able to help the mitochondria to enter the cells, but the carrier of the methods, such as the cell penetrating peptide or the liposome, may induce the mitochondria rupture and the cell membrane rupture, which damages the mitochondria and being toxic to target cells.

### SUMMARY

A main purpose of the present disclosure is providing a composition including an effective amount of exogenous mitochondria.

Another purpose of the present disclosure is providing a use of the composition, which is for repairing damaged mitochondria or reducing cell aging.

Another purpose of the present disclosure is providing a cell repairing method, including administrating an effective amount of exogenous mitochondria into a individual so that the exogenous mitochondria are completely sent into a cell so as to achieve the effects of repairing damaged cells, reducing or preventing cell aging.

To achieve the above purposes, an embodiment of the present disclosure discloses a composition including exogenous mitochondria and at least one pharmaceutically or cosmetically acceptable carrier.

Preferably, the composition further includes an adjuvant, and the adjuvant is selected from a group consisting of serum, plasma, complement and a combination of at least two of the above ingredients.

Preferably, the exogenous mitochondria are extracted from cells

Preferably, the exogenous mitochondria are obtained from cell by a centrifugal purification method.

In another embodiment of the present disclosure, a use of the above pharmaceutical composition is for reducing or preventing aging of skin cells.

In yet another embodiment of the present disclosure, a use of the above pharmaceutical composition is for repairing damaged cells.

A cell repairing method disclosed in an embodiment of the present disclosure is administrating an effective dosage of exogenous mitochondria into an individual so that the exogenous mitochondria enter a cell and substitute aged mitochondria or damaged mitochondria.

Preferably, before the exogenous mitochondria are administrated into the individual, the exogenous mitochondria are pretreated by at least one ingredient selected from a group consisting of serum, plasma, and complement.

The beneficial effects of the present disclosure are:

first, using the exogenous mitochondria can overcome the rejection induced by the allogeneic cell transplantation in prior arts;

second, the exogenous mitochondria are able to be obtained from normal cell line or living individual, which is a wide range of source and not hurtful to human health, for example, the known cell transplantation technique may lead to the occurrence of cancer or tumor;

third, the exogenous mitochondria are able to enter the cells directly and fuse with the endogenous mitochondria so as to substitute the damaged mitochondria in aged cells or damaged cells, and achieve the effects of reducing the oxidative stress of the cells, restoring the normal function of the cells, and being able to provide long-term and directly protection to the cells;

fourth, after the exogenous mitochondria are treated with the serum or the complement, it is able to enter the cells completely, and it is able to avoid the cytotoxic effect induced by the cell penetrating peptide or the liposome treatment; and

fifth, the exogenous mitochondria are able to fundamentally reduce wrinkles and skin aging phenomena and effectively induce the increase of collagen synthesis.

Accordingly, the pharmaceutical composition disclosed in the present disclosure shows high safety, and by administrating an effective dosage of pharmaceutical composition into an individual, as the exogenous mitochondria enter a cell, the effects of repairing the cell with damaged mitochondria and reducing the aging phenomena are achieved.

### BRIEF DESCRIPTION OF THE DRAWINGS

FIG. 1 shows the relative location of the engulfed mitochondria and the lysosome in the cells after the red fluorescent labeled mitochondria and the green fluorescent LysoTracker stained BHK cells are co-cultured for 1 hour;

FIG. 2 shows the relative location of the engulfed mitochondria and the lysosome in the cells after the red fluorescent labeled mitochondria and the BHK cells are co-cultured for 4 hours;

FIGS. 3A and 3B show observations of the exogenous mitochondria engulfed by the cell with a scanning electron microscope, wherein the square frames show the mitochondria being engulfed at low magnification; the arrows point out the mitochondria being engulfed by pseudopodia of the cell at high magnification;

FIG. 4 shows the observation result of the mitochondria enter the BHK cells, wherein the white bar represents 10 μm after the red fluorescent labeled mitochondria and the phalloidin-FITC stained BHK cells are co-cultured for 4 hours;

FIG. 5 shows the result of the red mitochondria enter the BHK cells after the BHK cells are treated with actinomycin D (ActD), the white bar represents 10 μm;

FIG. 6 shows the statistical results of the ratio of the cells with exogenous mitochondria in FIG. 4 and FIG. 5 which are BHK cells treated or not treated by AcD;

FIG. 7A shows the relative location of the engulfed mitochondria and the mitochondria in the cells after the no complement-treated red exogenous mitochondria and the BHK cells with green fluorescent mitochondria are co-cultured for 4 hours, wherein the arrow points the yellow fluorescent which represents the locations of the exogenous mitochondria and the endogenous mitochondria are overlapped in the cell;

FIG. 7B shows the relative location of the engulfed mitochondria and the mitochondria in the cells after the exogenous mitochondria treated by 10 μg/mL C3 complement and the BHK cells with green fluorescent mitochondria are co-cultured for 4 hours, wherein the arrows point the yellow fluorescent which represents the locations of the exogenous mitochondria and the endogenous mitochondria are overlapped in the cell;

FIG. 7C shows the observation and analysis of the red liner area in FIG. 7B by scanning confocal microscope, which represents the overlapped situation of the red fluorescent signal representing the exogenous mitochondria and the green fluorescent signal representing the endogenous mitochondria;

FIG. 8A shows the outward appearance of the untreated mitochondria observed by electron microscope;

FIG. 8B shows the outward appearance of the serum treated mitochondria observed by electron microscope;

FIG. 8C shows the outward appearance of the C3 complement treated mitochondria observed by electron microscope;

FIG. 8D shows the outward appearance of the Pep-1 cell penetrating peptide treated mitochondria observed by electron microscope;

FIG. 9A shows the result of the mitochondria enter the HUVEC cell after the exogenous mitochondria and the HUVEC cell are co-cultured;

FIG. 9B to 9D show the results of each of the HUVEC cell groups which are treated in different means after SA β-gal staining;

FIG. 10 shows the observation of the exogenous mitochondria with red fluorescent protein enter the dermis firoblast cell of mouse by scanning confocal microscope;

FIG. 11A to 11D show the images of the skin surfaces of the first to fourth groups of nude mouse after treated by exogenous mitochondria, which are observed by microscope;

FIG. 12 shows the analysis result of the roughness of the skin wrinkle of the nude mouse in each of the groups after treated by exogenous mitochondria; and

FIG. 13A to 13D show the result of the skin tissue sections of the nude mouse in each of the groups after stained by Masson's trichome.

### DETAILED DESCRIPTION

Unless otherwise defined, the meanings of the technical and scientific terms in the specification and the claims of are the same as the general meaning which the person with the ordinary skilled in the art of the present disclosure understands. If there is a contradictory situation, the meanings in the present disclosure are taken as the basis.

The term “effective dosage” means the required dosage of the compound or the active component for generating specific effect, which is able to be represented by the weight percentage in the composition. As known by person ordinary skilled in the art of the present disclosure, the effective dosage is varied according to the administration route for generating the specific effect. Generally, the dosage of the active component or the compound in the composition in weight percentage is able to be 1% to 100%, and more preferably is about 30% to 100%.

The term “pharmaceutically or cosmetically acceptable carrier” includes the carrier used in the medical product or the cosmetic product of any standard, and the carrier is able to be solid, semi-solid or liquid according to the form of the composition. For example, the carrier includes but not limited to gelatin, emulsifier, hydrocarbon mixture, water, glycerol, physiological saline, buffered saline, lanolin, paraffin, beeswax, dimethyl silicone oil, and ethanol.

The term “composition” includes an effective amount of the compound or the active component for generating specific effect, and at least one carrier. As known by the person with the ordinary skill in the art of the present disclosure, the form of the composition is able to be varied according to the administration route for inducing the specific effect, such as lozenge, powder, injection, etc., and the carrier is also able to be solid, semi-solid or liquid according to the form of the composition.

The term “administrate” means a route to deliver an object to a specific part of an individual, a specific cell, a specific target or a means to contact a individual. Generally, the administration route includes but not limited to oral, smear, spray, inhalation, injection, etc.

In the following, for further explaining the effect of the present disclosure, several embodiments are illustrated in detail. However, these embodiments are examples for explanation, and any phrase used in the explanation does not restrict the scope and the meaning of the specification and the claims of the present disclosure.

### Embodiment One: Fluorescent Labeled Mitochondria

Transfect the red fluorescent proteins DsRed, which carry the mitochondria signal peptide, or the green fluorescent into the baby hamster kidney fibroblast cells (BHK-21 cells) so as to obtain the RedM-BHK cells or GFP-BHK cells which is able to continuously express the red fluorescent protein by screening with G418 antibiotics and flow cytometer.

### Embodiment Two: Separate Mitochondria from BHK Cells

When the cell number of the cultured BHK cells reaches 2×10⁸, the SEH buffer (0.25 M sucrose, 0.5 mM EGTA and 3 mM HEPES-NaOH, pH 7.2) is added to wash the cell culture dish, and centrifuge it at 1000×g for 3 minutes. After removing the supernatant from it, add 2 mL of SEH buffer into it, and it is grind about 15 times in a Dounce homogenizer, and the operation is performed on ice to reduce the damage to cells and mitochondria. After grinding is finished, centrifuge the homogenate solution at 1000×g for 15 minutes to remove the precipitate, and then centrifuge it at 9000×g for 10 minutes, and at last, after the final precipitate is dissolved in 50 μL of SEH buffer, the protease inhibitor is added into it, and it is stored at 4° C.

### Embodiment Three: Confirm the Route for the Mitochondria to Enter the Cells

In this embodiment, to track the moving route for the mitochondria to enter the cells, the exogenous mitochondria are added to observe the location of the moving mitochondria and the lysosomes at different times.

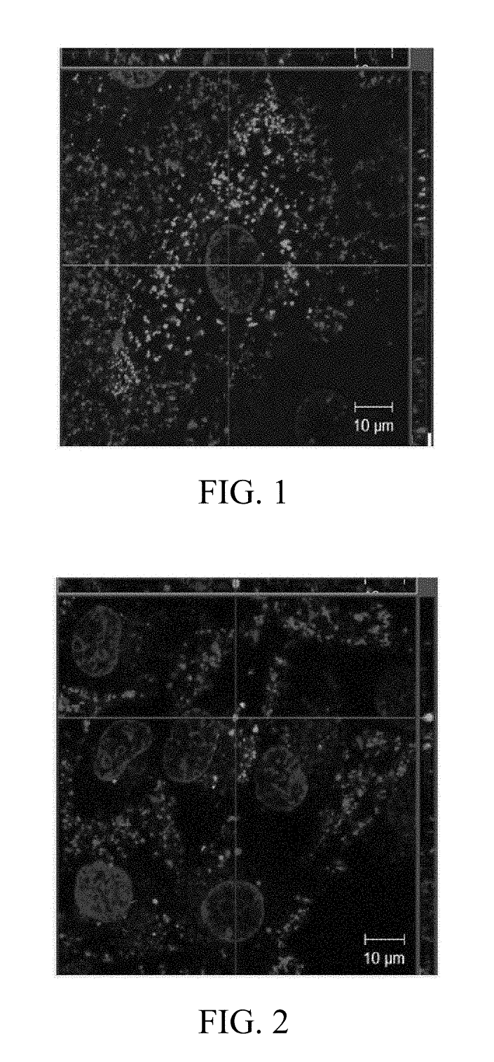

First, label the mitochondria by the red fluorescent protein DsRed, and transfect the BHK cells by the LysoTracker showing green fluorescent so as to confirm the location of the lysosomes in the cell. Take 5 μg exogenous mitochondria, which are labeled by red fluorescent protein, and the LysoTracker treated BHK cells, and co-culture them at room temperature. At the time of culturing for one hour and four hours, observe the situation of the exogenous mitochondria enter the BHK cells and the relative location of the exogenous mitochondria and the lysosomes by scanning confocal microscope, and the results are shown in FIG. 1 and FIG. 2.

As shown in FIG. 1, after culturing the exogenous mitochondria which shows red fluorescent for one hour, the exogenous mitochondria are distributed around the BHK cell. As shown in FIG. 2, after culturing for four hours, a part of the exogenous mitochondria, which shows red fluorescent, and the green fluorescent of the LysoTracker signals are overlapped. According to the above result, the exogenous mitochondria and the lysosomes are located at the same location in the cell after the exogenous mitochondria enter the cell, and thus, it is inferred that the exogenous mitochondria enter the cell by phagocytosis.

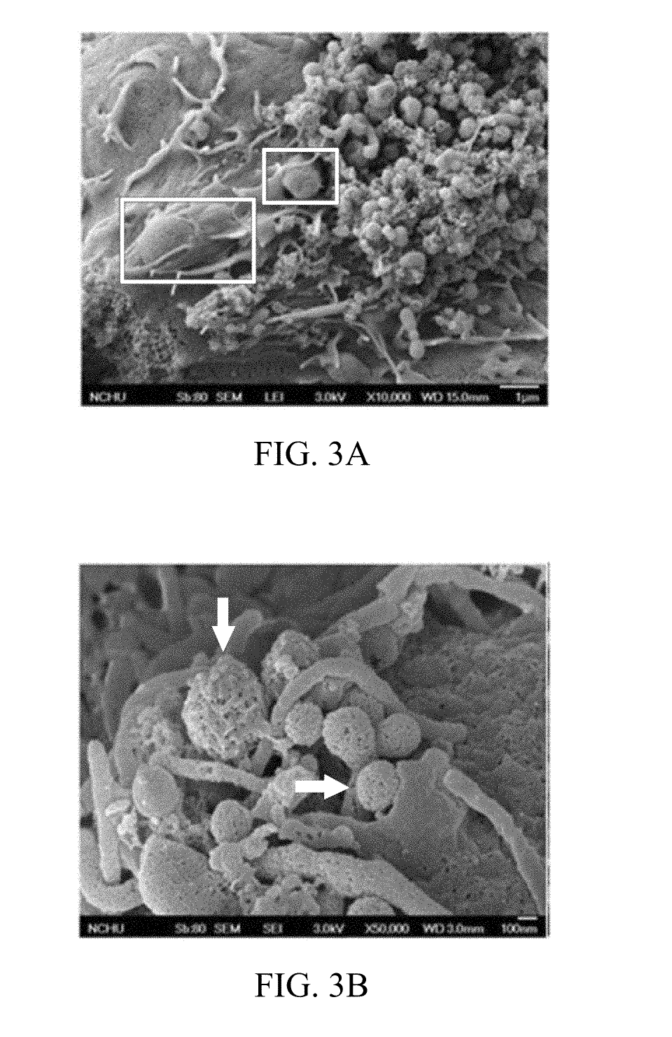

The situation of the exogenous mitochondria that enter the BHK cells is further observed by scanning electron microscope. As shown in FIG. 3A and 3B, FIG. 3A is the observation result at low magnification, and the square frames in it show the mitochondria being engulfed by the cells; FIG. 3B is the observation result at high magnification, and the arrows in the figure point out the mitochondria being engulfed by pseudopodia of the cell. Therefore, the results in FIG. 3A and 3B show that the BHK cells cover the exogenous mitochondria by extending the pseudopodia, which confirms the route for the exogenous mitochondria to enter the cells is phagocytosis.

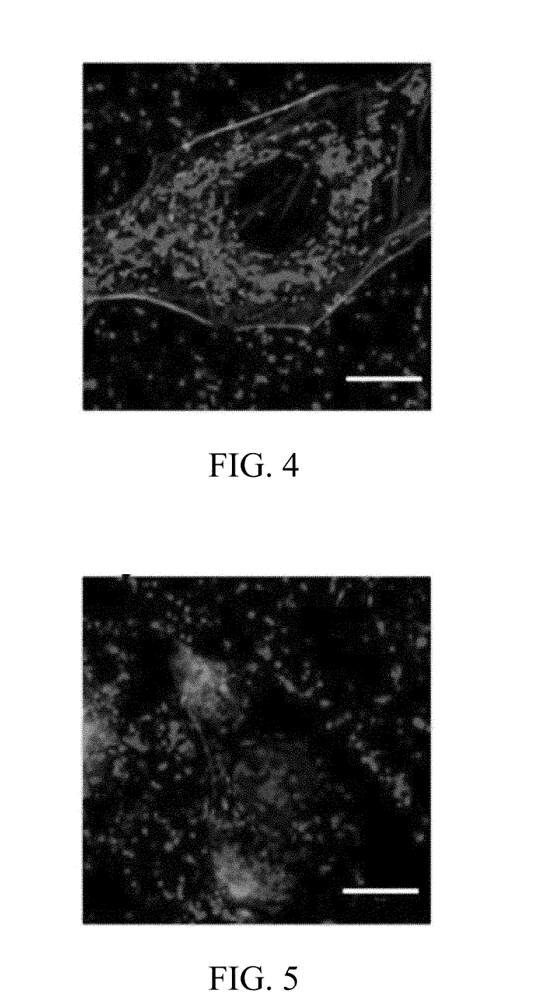

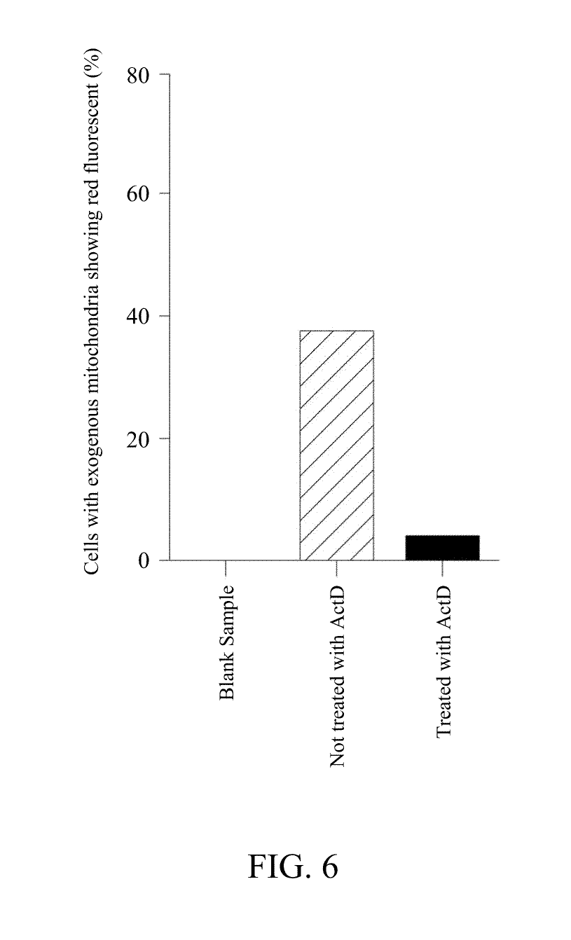

Moreover, stained the BHK cells by phalloidin-FITC to label the actin in the cell and show the type of the cell. After culturing the stained BHK cells and the red fluorescent labeled exogenous mitochondria at 37° C. for four hours, it is able to be found that a lot of mitochondria enter the cell, as shown in FIG. 4. However, treat the BHK cells with 20 μM actinomycin D (ActD) so that the phagocytosis of the BHK cells is inhibited, and it is able to be found that the exogenous mitochondria are totally unable to enter the cell, as shown in FIG. 5. Calculate the number of the exogenous mitochondria that enter the above cells which are treated in different means, and the result is shown in FIG. 6.

According to the above results, when simply provide the exogenous mitochondria to the cells, the cells engulf the mitochondria by phagocytosis so that the mitochondria are able to enter the cell.

### Embodiment Four: Serum is Helpful for Mitochondria to Enter Cells

Take diluted fetal bovine serum (GIBCO) and mix the exogenous mitochondria, which are labeled by red protein, and serum for one hour, then remove the serum in the supernatant by centrifugation, and then dissolve the precipitate by the SHE buffer to restore the original volume. Stain the BHK cells by phalloidin-FITC to define the interface of the cell membrane by the specific binding between the stain and the F-actin.

The BHK cells are divided into four groups, wherein, group I is blank sample; group two is mixed with the serum which is diluted 1000 times; group three is mixed with the serum which is diluted 500 times; group four is mixed with the serum which is diluted 100 times. Extract the mitochondria from the above-mentioned SEH solution, and culture the mitochondria and the BHK cells of each group at 37° C. for 4 hours. Then observe the situation of the mitochondria, which show red fluorescence, enter the cell by laser conjugation focusing microscope, and analysis the number of single cell containing the red mitochondria. The result is shown in Table 1 below, and Table 1 is analyzed by the one-way ANOVA test method. The asterisk indicates that the p value is less than 0.05, which represents a statistically significant difference from group I, the blank group.

TABLE 1
Ratio and average number of the exogenous
mitochondria in each group of the BHK cells

| | Group | | | |
|---|---|---|---|---|
| | Group I | Group II | Group III | Group IV |
| cell with | 28.2 ± 3.4 | 43.4 ± 7.5* | 50.4 ± 5.2* | 64.3 ± 8.6* |
| exogenous | | | | |
| mitochondria/ | | | | |
| total cell | | | | |
| number (%) | | | | |
| Average | 6.2 ± 1.8 | 6.8 ± 3.3 | 7.8 ± 6.8* | 12.8 ± 5.9* |
| exogenous | | | | |
| mitochondria | | | | |
| number in each | | | | |
| cell (number) | | | | |

According to the result in table 1 above, in the presence of serum, the number of cells with exogenous mitochondria and the number of exogenous mitochondria enter a single cell are significantly higher than those without serum treatment. It can be known that treating the exogenous mitochondria or cell by serum helps to increase the efficiency of the exogenous mitochondria to enter cells.

### Embodiment Five: Complement is Helpful for Mitochondria to Enter Cells

Mix the red fluorescent protein labeled exogenous mitochondria and C3 complements with a predetermined concentration for one hour, and centrifuge it to remove the C3 complement from the supernatant, and then dissolve the precipitated exogenous mitochondria by the SHE buffer to restore the original volume.

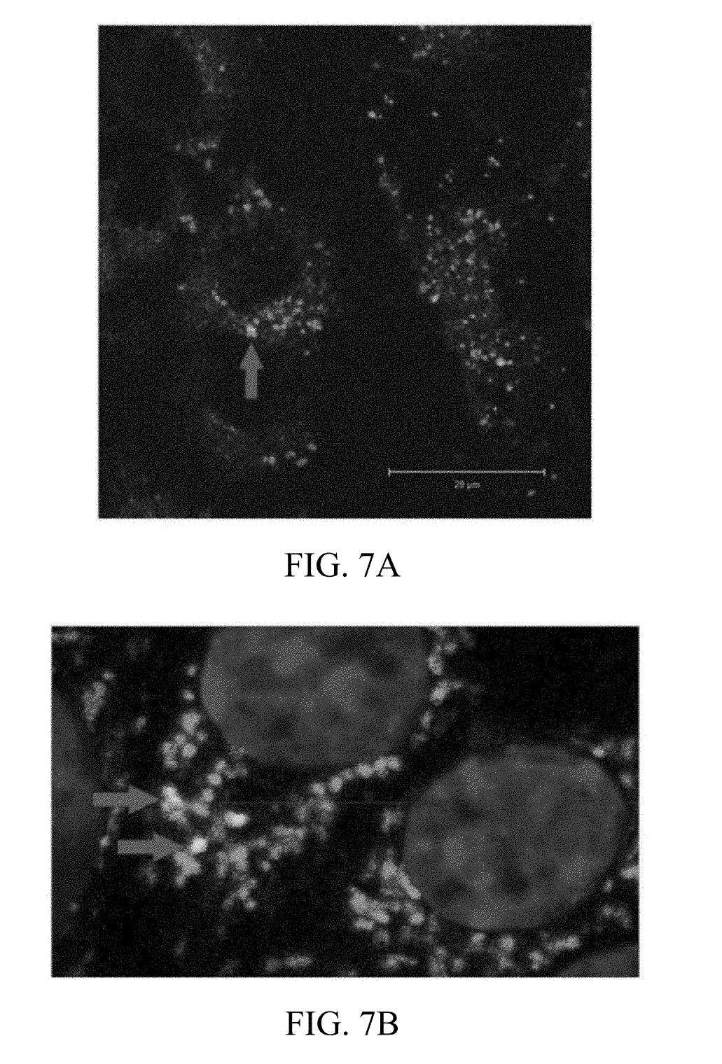

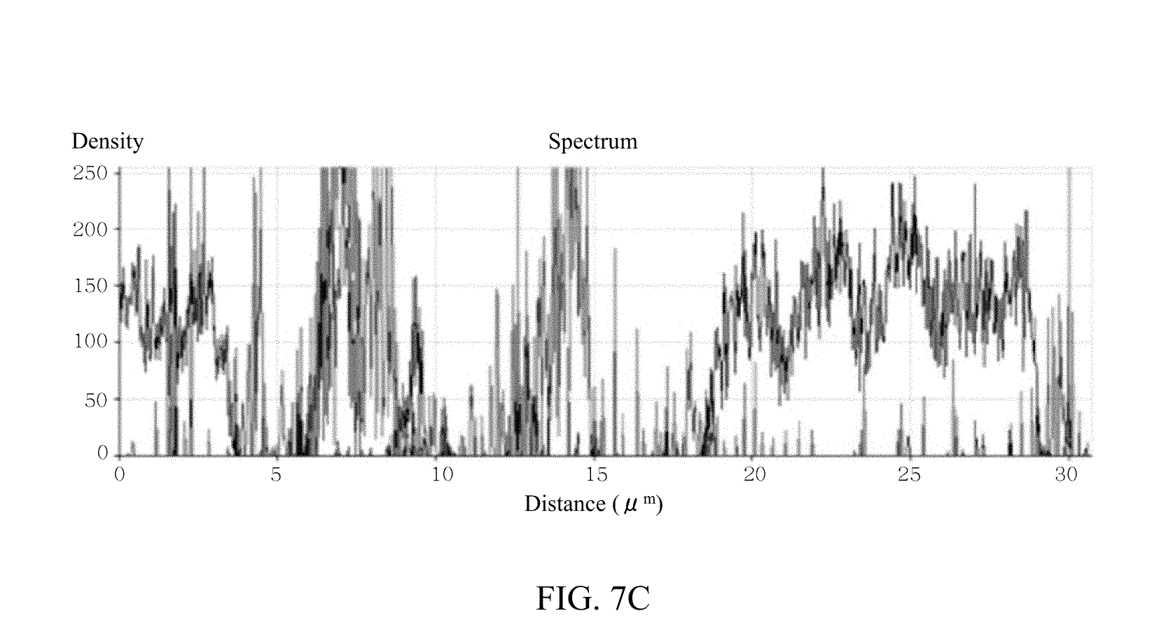

Group I is untreated sample. Group two to group five are 5 μg exogenous mitochondria treated by the C3 complement (Sigma-Aldrich) in the concentration of 0.1 μg/mL, 1 μg/mL, 10 μg/mL and 20 μg/mL, respectively. After co-culturing at 37° C. for 4 hours, observe the red fluorescence in the cells of each of the groups by laser conjugation focusing microscope, and calculate the ratios of the cell in each of the groups containing the exogenous mitochondria, and then perform quantitative statistics. The fusion situation of the endogenous mitochondria and the exogenous mitochondria after the exogenous mitochondria enter the cells are shown in FIGS. 7A to 7C.

Please refer to FIGS. 7A and 7B, the arrows in the figures point to the yellow fluorescent which represents the exogenous mitochondria and the endogenous mitochondria are overlapped in the cells. Therefore, according to the result in FIGS. 7A to 7C, no mater the exogenous mitochondria are treated by the complement or not, the exogenous mitochondria engulfed by the cells and the original mitochondria in the cells are located at the same position in the cells, which shows there is the fusion phenomenon between the exogenous mitochondria and the endogenous mitochondria, and the exogenous mitochondria are able to escape from the phagolysosome and enter the cytoplasm.

Moreover, according to the analysis result of the flow cytometer, in group I which is not treated by the C3 complement, an average of about 26.16±4.75% of the cells are detected with the red fluorescent; an average of about 43.43±3.5% of the cells are detected with the red fluorescent in group II; an average of about 65.13±7.5% of the cells are detected with the red fluorescent in group III; an average of about 78.97±13.35% of the cells are detected with the red fluorescent in group IV; about 80% of the cells are detected with the red fluorescent in group V; moreover, group II to group V show statistically significant difference from group I (p<0.05).

According to the above results, by providing complement to the exogenous mitochondria, the ratio of the exogenous mitochondria, which enter the cells, is significantly increased, and the engulfed exogenous mitochondria are able to fuse with the endogenous mitochondria. In addition, as the concentration of the complement is increased, the number of the exogenous mitochondria, which enter the cells, is also increased.

### Embodiment Six: the Separated Mitochondria are not Damaged by Serum or Complement

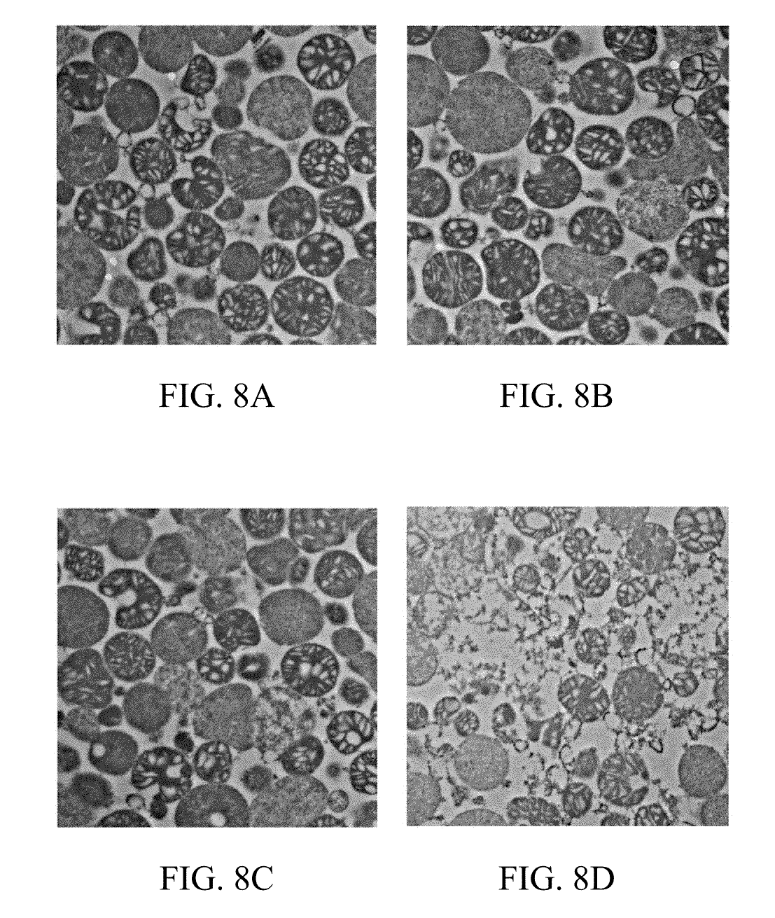

Divide the separated exogenous mitochondria into four groups, and each group is 5 μg. Wherein, group I is blank sample; group II is the exogenous mitochondria treated by fetal bovine serum which is diluted 100 times; group III is the exogenous mitochondria treated by the C3 complement in the concentration of 10 μg/mL; group IV is the exogenous mitochondria treated by cell penetrating peptide Pep-1 in the concentration of 100 nM. Each of the groups is cultured at 37° C. for 4 hours. Then observe the outward appearance of the mitochondria in each group by transmission electron microscope; the results are shown in FIGS. 8A to 8D.

According to the results in FIGS. 8A to 8D, the outward appearance of the mitochondria in group II and group III are similar to the outward appearance of the mitochondria in group I. Comparing with group I, the exogenous mitochondria in group IV, which are treated by cell penetrating peptide, are swollen and have rupture. Therefore, comparing with the cell penetrating peptide, the serum and the complement show lower toxicity and would not damage the outward appearance so that they are able to maintain the completeness of the mitochondria after the mitochondria enter the cells.

### Embodiment Seven: Culture Human Umbilical Vascular Endothelial Cells

Human umbilical vascular endothelial cells (HUVEC cell) are bought from Food Industry Research and Development Institute in Hsinchu. The HUVEC cells are cultured in M199 medium, and 10% fetal bovine serum, 0.1% heparin and 0.03% endothelial cell growth supplement are added. The HUVEC cells are able to be cultured on the 0.1% by weight gelatin-coated petri dishes.

### Embodiment Eight: Mitochondria Retard Cell Aging

First, separate mitochondria from human fibroblast HS68, and the mitochondria are used as the source of the exogenous mitochondria. Each group is 5 μg. After treating the mitochondria by complement, stain the mitochondria by the mitochondria tracking stain (Mitotracker) which shows red fluorescent.

Then, treat the first generation of the cultured HUVEC cells with hydrogen peroxide to age it. The eight generation of the cultured HUVEC cells (8×10⁵ cell/well) are treated by the 100 μM hydrogen peroxide at 37° C. for 2 hours, and wash them by phosphate buffer to remove the hydrogen peroxide. After culturing them in normal cell culture medium for one day, divide them into three groups; wherein, group I is a blank group which has no mitochondria being added; group II is the mitochondria which are not treated by complement; group III is the mitochondria which are treated by complement. After the cells in each of the groups are respectively cultured for four hours, perform the Senescence-associated β-galatosidase (SA β-gal) stain to them, respectively, and the Ki67 and BrdU stain analysis.

The stain processes of Ki67 and BrdU is the general well-known technique in the art of the present disclosure and is generally known to those skilled in the art, and therefore, the stain processes are not repeat here.

The process of the SA β-gal stain are shown in the following: firstly, wash the cells by phosphate buffer, and then perform the fixiation by 2% paraformaldehyde and 0.2% glutaraldehyde for five minutes, and then treat them by stain at 37° C. for 12 hours, wherein the stain includes 1 5-bromo-4-chloro-3-indolyl-β-D-galactoside (BCIG or X-gal), 40 mM citric acid/phosphate buffer (pH 6.0), 5 mM potassium ferricyanide, 5 mM sodiumferricyanide, 150 mM NaCl, and 2 mM MgCl₂. At last, stain the cells by 0.5% Eosin, and observe them by microscope.

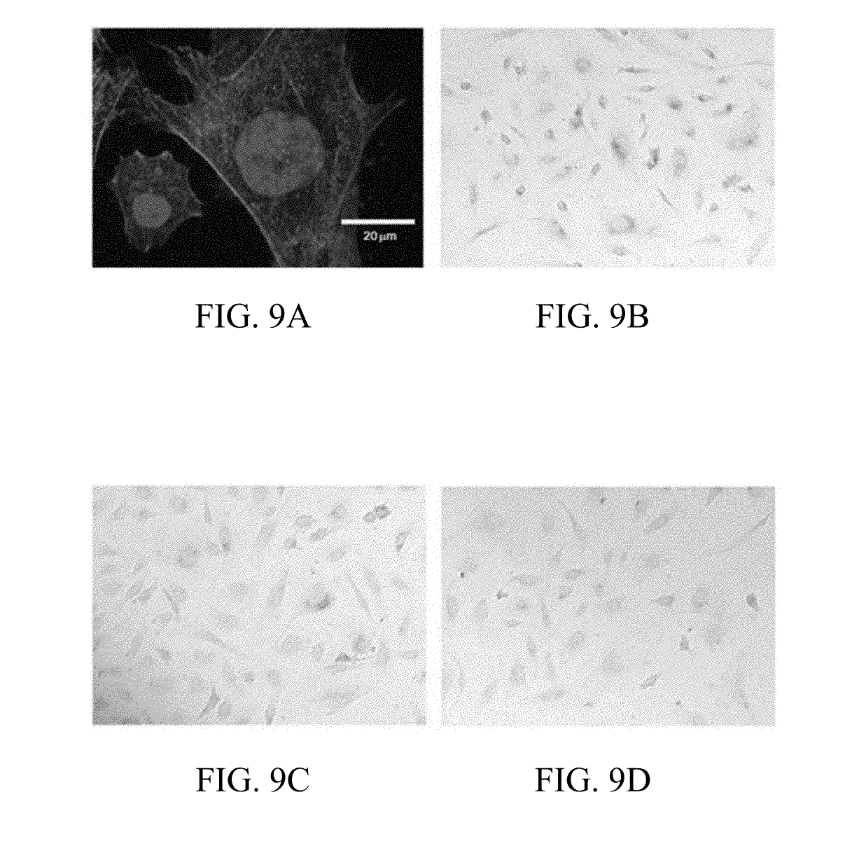

The results after the SA β-gal stain are shown in FIGS. 9A to 9D. As shown in FIG. 9A, the exogenous mitochondria are able to enter the HUVEC cells. As shown in FIGS. 9B to 9D, the HUVEC cells in group I are stained by SA β-gal obviously. Although the HUVEC cells in group II are stained by the SA β-gal, but comparing with group I, the number of the stained HUVEC cells in group II is significantly decreased. Comparing with group I and group II, the HUVEC cells in group III are almost not stained by SA β-gal. Perform further statistical analysis of the stained result, it shows that about 85±12.3% cells in group I are stained, and about 60.1±6.8% cells in group II are stained, and about 25±6.2% cells in group III are stained.

Moreover, after performing counting to the Ki67 and the BrdU stain results, the ratio of the HUVEC cells in group I which are stained by Ki67 and BrdU are 13.3% and 13%, respectively. Comparing with group I, the ratio of the HUVEC cells in group II which are stained by Ki67 and BrdU are increased to 35% and 33%, respectively. The ratio of the HUVEC cells in group III which are stained by Ki67 and BrdU are the highest, and they are 71% and 59.6%, respectively. Moreover, when the added mitochondria are treated by fetal bovine serum, the same effect as treated by complement is able to be achieved.

According to the above result, the exogenous mitochondria enter the cells are able to reduce the degree of cell aging, improve cell growth and improve cell duplication efficiency. Moreover, with the number of the exogenous mitochondria enter the cells is increased, the degree of cell aging is significantly decreased, and the number of cell at cell division state is increased so as to improve cell duplication and growth. Accordingly, administrating the pharmaceutical composition, which includes exogenous mitochondria, disclosed in the present disclosure to an individual is able to reduce or retard the degree of cell aging, and when the pharmaceutical composition further includes the composition, which is helpful for the mitochondrial to enter the cell, such as serum, plasma or complement, the effect is significantly improved.

### Embodiment Nine: Animal Experiment (1)

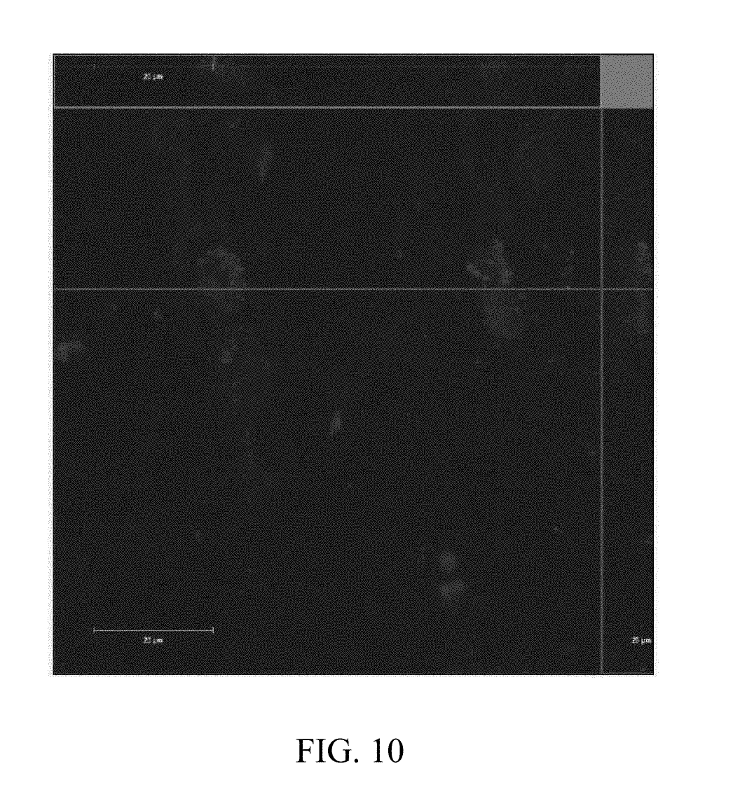

Separate the mitochondria with red fluorescent from RedM-BHK cells, and treat by fetal bovine serum which is diluted 100 times or 10 μg/mL C3 complement. Take the 48-week-old natural aging nude mice, and inject the mitochondria into the subcutaneous tissue of the nude mice. After an hour, take the nude mouse's whole skin, and then perform fixiation with 4% paraformaldehyde for 5 minutes, and then place the sample in 0.1M phosphate buffer until the sample sinking, and then infiltrate and mount the sample by optimal cutting temperature compound (OCT) to perform frozen section, and the thickness of the section is 12 μm. Observe the section by conjugation focusing microscope, and the result is shown in FIG. 10.

FIG. 10 shows the dermis area of the nude mice after the mitochondria transplantation. The blue fluorescent represents the nucleus in the fibroblast stained by DAPI, and the red fluorescent represents the mitochondria separated from the RedM-BHK cells. As shown in FIG. 10, the mitochondria are able to enter the fibroblast in the dermis after transplantation.

### Embodiment Ten: Separate Mitochondria from Hypatocyte

First, the mice were sacrificed after deep anesthesia, and perform the perfusion at the mice by physiological saline until the blood in the liver is removed. Take 1 cm³ liver tissue, and add about 6 ml SEH buffer to perform grinding by tissue grinder, and then centrifuge it at 1000×g for 15 minutes to obtain the supernatant. Moreover, at the same time, add sucrose solution with the concentration of 55%, 40% and 30% into the centrifuge tube to obtain the 55% sucrose gradient centrifuge tube. Add the supernatant obtained in the centrifugation process to the top layer of the gradient centrifuge tube, and then centrifuge it at 35000 rpm for 30 minutes to form a white layer at the interface between the 40% layer and the 55% layer. About 1 ml of the white layer is collected and added into a 15 ml centrifuge tube, and 5 ml SEH buffer is added to centrifuge it at 13000×g for 3 minutes, and then remove the supernatant, and then repeat the centrifugation process for 3 times. At last, after the mitochondria precipitate is dissolved in 200 μL SEH buffer, the protease inhibitor is added into it, and it is stored at 4° C.

### Embodiment Eleven: Animal Experiment (2)

Take and divide thirty-two 48-week-old natural aging nude mouse into four groups, each of the groups has eight mouse and treated in different condition for twelve weeks, wherein group I is non-treatment group, and 1000 μg hypatocyte mitochondria are injected into each mice in group II every week, and 1000 μg hypatocyte mitochondria, which are treated by complement, are injected into each mice in group III every week, and 1000 μg hypatocyte mitochondria, which are treated by serum, are injected into each mice in group IV every week. The subcutaneous injection method for the mouse in group II to group IV is that 5000 μg/mL hypatocyte mitochondria are divided equally to be injected to twenty points on the back of each mice, and the injection dosage at each point is 0.01 ml, and the total injection dosage is 0.2 ml. In order to remove the effect on the wrinkle only caused by complement or serum, before injecting the complement or the serum treated mitochondria, the complement and the serum remained in the supernatant are removed by high speed centrifugation twice in the experiment.

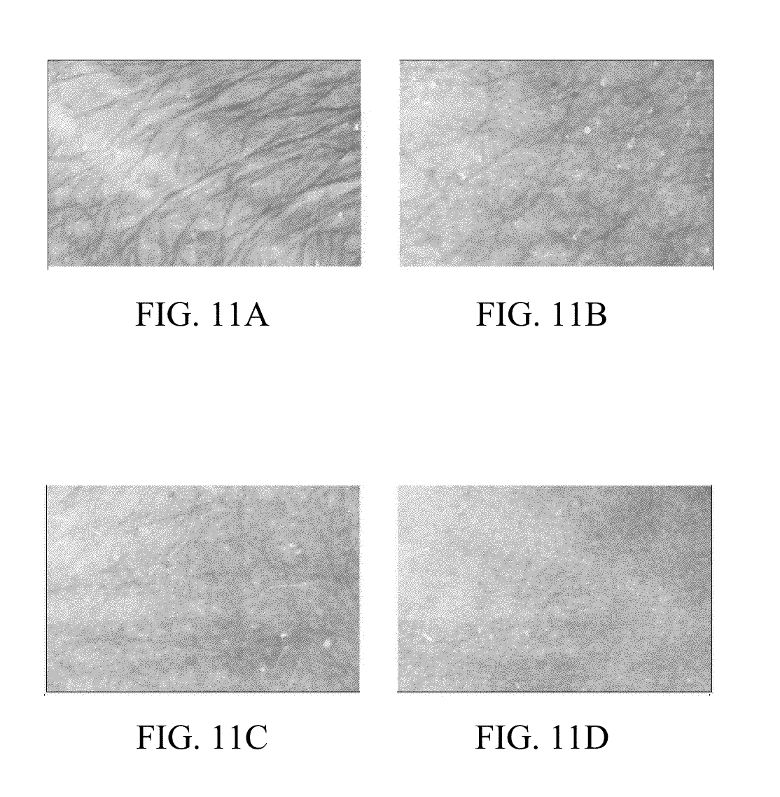

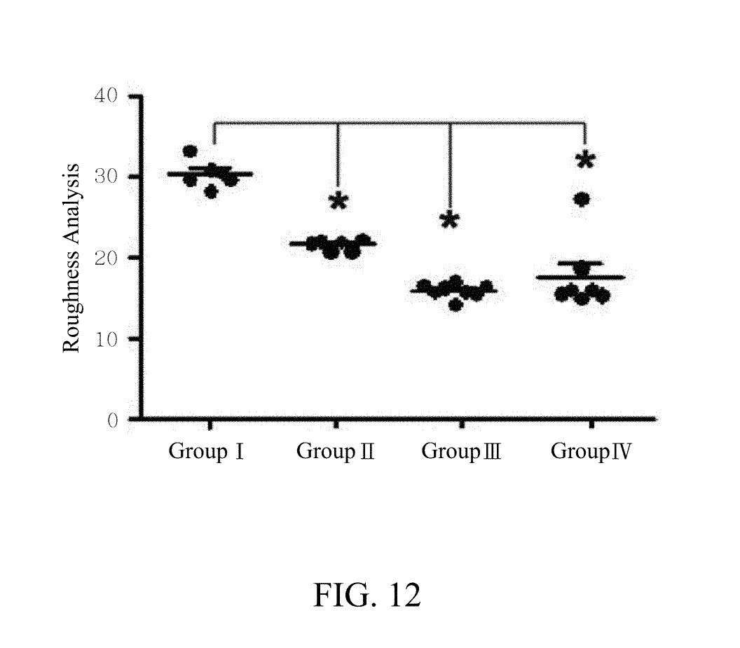

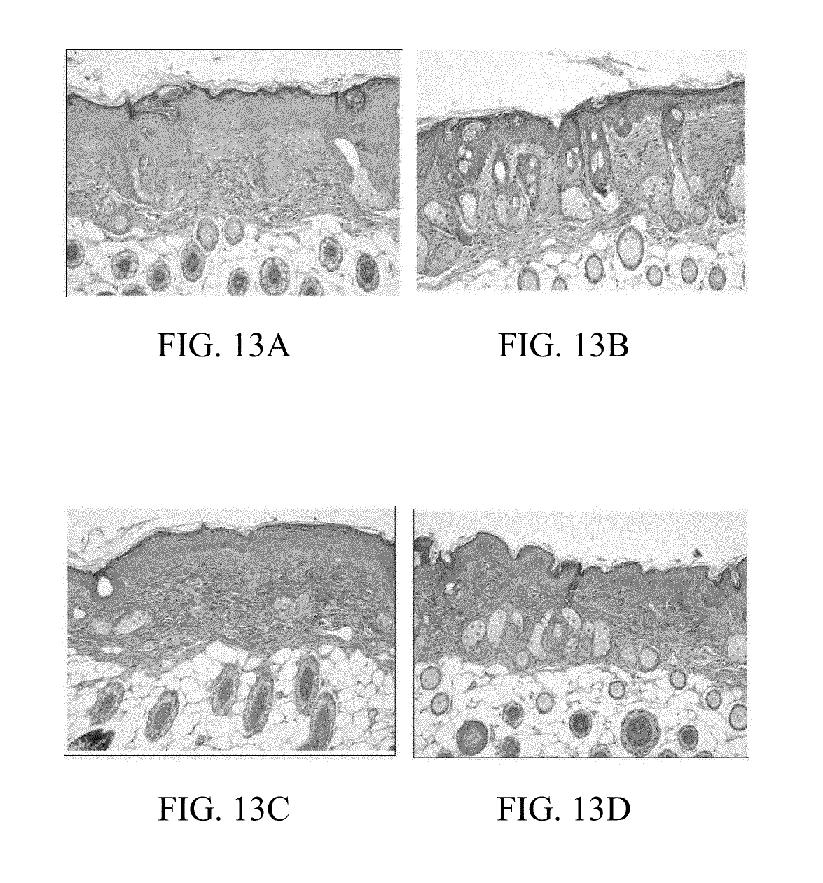

After the experiment, as the process shown in embodiment nine, the whole skin of each nude mice in each group is photographed, frozen sections and stained. The photograph results of the skin are shown in FIGS. 11A to 11D, and the roughness of the epidermal wrinkle of the nude mouse in each group is analyzed, and the result is shown in FIG. 12, wherein, * represents a significant difference from group I. Moreover, the skin tissues of the nude mouse in each group are stained with Masson's trichrome to show the content of the dermal collagen layer, and the results are shown in FIGS. 13A to 13D.

As the result shown in FIG. 12, the wrinkles observed in group II to group IV, which have exogenous mitochondria injections, are lower than that in group I, which has no treatment; wherein the wrinkles on the skin of the nude mouse in group III and group IV are lighter than that in group II. Moreover, as the result shown in FIGS. 13A to 13D, group has the most thick epidermis, and the thickness of the epidermis in group II to group IV are thinner than that in group I, and the staining of the collagen layer are deeper than that in group I.

According to the result shown in FIGS. 11A to 13D, the administrated exogenous mitochondria disclosed in the present disclosure are able to enter the cells of the living body, effectively reduce the wrinkle generation and improve the collagen generation ability of the epidermal fibroblast. Moreover, since the serum and the complement are helpful for the exogenous mitochondria to enter cells, the exogenous mitochondria treated by serum or complement have better anti aging ability. Accordingly, the pharmaceutical composition, which includes exogenous mitochondria, disclosed in the present disclosure is able to achieve the effect of retarding or reducing skin aging.

According to the above embodiments, the exogenous mitochondria and the pharmaceutical composition including the same as the active component disclosed in the present disclosure have the following benefits:

first, using the exogenous mitochondria can overcome the rejection induced by the allogeneic cell transplantation in prior arts;

second, the exogenous mitochondria are able to be obtained from normal cell line or living individual, which is a wide range of source and not hurtful to human health, for example, the known cell transplantation technique may lead to the occurrence of cancer or tumor;

third, the exogenous mitochondria are able to enter the cells directly and fuse with the endogenous mitochondria so as to substitute the damaged mitochondria in aged cells or damaged cells, and achieve the effects of reducing the oxidative stress of the cells, restoring the normal function of the cells, and being able to provide long-term and directly protection to the cells;

fourth, after the exogenous mitochondria are treated with the serum or the complement, it is able to enter the cells completely, and it is able to avoid the cytotoxic effect induced by the cell penetrating peptide or the liposome treatment; and

fifth, the exogenous mitochondria are able to fundamentally reduce wrinkles and skin aging phenomena and effectively induce the increase of collagen synthesis.

Accordingly, the pharmaceutical composition disclosed in the present disclosure shows high safety, and by administrating an effective dosage of pharmaceutical composition into an individual, as the exogenous mitochondria enter a cell, the effects of repairing the cell with damaged mitochondria and reducing the aging phenomena are achieved.

The above is only illustrations to the present disclosure by the embodiments, and any change or amendment to the embodiments in the specification within the scope of the spirit of the present disclosure by the person having the ordinary skill in the art should be covered by the claims of the present disclosure.

### REFERENCE

Lamb C A, Yoshimori T, Tooze S A (2013) The autophagosome: origins unknown, biogenesis complex. Nat Rev Mol Cell Biol 14: 759-774.

Mukhopadhyay S, Panda P K, Sinha N, Das D N, Bhutia S K (2014) Autophagy and apoptosis: where do they meet? Apoptosis 19: 555-566.

Ghavami S, Shojaei S, Yeganeh B, Ande S R, Jangamreddy J R, et al. (2014) Autophagy and apoptosis dysfunction in neurodegenerative disorders. Prog Neurobiol 112:24-49.

Brennan L A, Kantorow M (2009) Mitochondrial function and redox control in the aging eye: role of MsrA and other repair systems in cataract and macular degenerations. Exp Eye Res 88: 195-203.

Jarrett S G, Lewin A S, Boulton M E (2010) The importance of mitochondria in age-related and inherited eye disorders. Ophthalmic Res 44: 179-190.

Blatt T, Lenz H, Koop U, Jaspers S, Weber T, et al. (2005) Stimulation of skin's energy metabolism provides multiple benefits for mature human skin. Biofactors 25:179-185.

Makrantonaki E, Zouboulis C C (2007) Molecular mechanisms of skin aging: state of the art. Ann N Y Acad Sci 1119: 40-50.

King M P, Attardi G (1988) Injection of mitochondria into human cells leads to a rapid replacement of the endogenous mitochondrial DNA. Cell 52: 811-819.

King M P, Attardi G (1989) Human cells lacking mtDNA: repopulation with exogenous mitochondria by complementation. Science 246: 500-503.

Chang J C, Liu K H, Li Y C, Kou S J, Wei Y H, et al. (2013) Functional recovery of human cells harbouring the mitochondrial DNA mutation MERRF A8344G via peptide-mediated mitochondrial delivery. Neurosignals 21: 160-173.

Spees J L, Olson S D, Whitney M J, Prockop D J (2006) Mitochondrial transfer between cells can rescue aerobic respiration. Proc Natl Acad Sci USA 103: 1283-1288.

What is claimed is:

1. A composition, comprising:
exogenous mitochondria; and
at least one pharmaceutically or cosmetically acceptable carrier.

2. The composition of claim 1, further comprising an adjuvant, and the adjuvant being selected from a group consisting of serum, plasma, complement and a combination of at least two of the above ingredients.

3. The composition of claim 1, wherein the exogenous mitochondria are extracted from cells.

4. The composition of claim 1, wherein the exogenous mitochondria are obtained from cell by a centrifugal purification method.

5. A use of the composition of claim 1 for reducing or preventing aging of skin cells.

6. A use of the composition of claim 1 for repairing a cell with damaged mitochondria.

7. A cell repairing method comprising administrating an effective dosage of exogenous mitochondria into an individual so that the exogenous mitochondria enter a cell and substitute aged mitochondria or damaged mitochondria.

8. The anti cell aging method of claim 7, wherein before the exogenous mitochondria are administrated into the individual, the exogenous mitochondria are pretreated by at least one ingredient selected from a group consisting of serum, plasma, and complement.
